(19) United States
(12) Patent Application Publication
(10) Pub. No.: US 2025/0078998 A1
(43) Pub. Date: Mar. 6, 2025

(54) SYSTEM AND METHOD FOR MENTAL HEALTH DISORDER DETECTION SYSTEM BASED ON WEARABLE SENSORS AND ARTIFICIAL NEURAL NETWORKS

(71) Applicant: The Trustees of Princeton University, Princeton, NJ (US)

(72) Inventors: Shayan HASSANTABAR, Ewing, NJ (US); Zhao ZHANG, Palo Alto, CA (US); Hongxu YIN, Princeton, NJ (US); Niraj K. JHA, Princeton, NJ (US)

(73) Assignee: The Trustees of Princeton University, Princeton, NJ (US)

(21) Appl. No.: 18/235,422

(22) PCT Filed: Feb. 1, 2022
(86) PCT No.: PCT/US2022/014697

§ 371 Date: Aug. 18, 2023

Related U.S. Application Data
(60) Provisional application No. 63/150,822, filed on Feb. 18, 2021.

(51) Int. Cl. G16H 50/20; G16H 40/67

(52) U.S. Cl. CPC G16H 50/20; G16H 40/67

(57) ABSTRACT

According to various embodiments, a machine-learning based system for mental health disorder identification and monitoring is disclosed. The system includes one or more processors configured to interact with a plurality of wearable medical sensors (WMSs). The processors are configured to receive physiological data from the WMSs. The processors are further configured to train at least one neural network based on raw physiological data augmented with synthetic data and subjected to a grow-and-prune paradigm to generate at least one mental health disorder inference model. The processors are also configured to output a mental health disorder-based decision by inputting the received physiological data into the generated mental health disorder inference model.

### CROSS-REFERENCE TO RELATED APPLICATIONS

This application claims priority to provisional application 63/150,822, filed Feb. 18, 2021, which is herein incorporated by reference in its entirety.

### STATEMENT REGARDING FEDERALLY SPONSORED RESEARCH OR DEVELOPMENT

This invention was made with government support under Grant No. CNS-1907381 awarded by the National Science Foundation. The government has certain rights in the invention.

### FIELD OF THE INVENTION

The present invention relates generally to wearable medical sensors and neural networks and more particularly to a system and method for identification and monitoring of mental illnesses based on wearable medical sensor data and neural network processing that bypasses feature extraction and generates synthetic data.

### BACKGROUND OF THE INVENTION

Mental health problems impact around 20% of the world population. They may negatively affect a person's mind, emotions, behavior, and even physical health. Mental health issues may include various disorders like bipolar, depression, schizophrenia, and attention-deficit hyperactivity, to name but a few. These disorders not only affect adults but children and adolescents as well. Moreover, patients with serious mental health issues have a higher risk of morbidity due to physical health problems.

In order to understand the mental health condition of the patient and provide suitable patient care, early detection is essential. However, this remains a public health challenge. While many other diseases can be diagnosed based on specific medical tests and laboratory measurements, detection of mental health problems mainly relies on self-reports and responses to specific questionnaires designed for identifying certain patterns of behavior and social interactions. Hence, to address this challenge, novel detection strategies are needed.

There has been recent interest in employing machine learning to detect mental health conditions. Neural networks (NNs) are popular machine learning models that use nonlinear computations to make inferences from large datasets. Thus, they have started being deployed in the smart healthcare domain.

In previous studies, two main data sources for deep learning-based analysis of mental health have been clinical data and social media usage data. The former includes studies that use neuro-image data for detecting various mental health disorders, electroencephalogram (EEG) data to study brain disorders, and electronic health records (EHRs) to study mental health problems. Moreover, social media usage patterns have been used to predict the personal traits of the user. As a result, several recent works focus on exploiting such patterns to detect psychiatric illness.

Although these works have demonstrated the promise of using machine learning in identifying mental health disorders, daily mental health monitoring is still a challenge. Since mental health condition treatment delays may lead to negative outcomes, potentially even loss of life, it is desirable to have immediate and pervasive mental health detection.

### SUMMARY OF THE INVENTION

According to various embodiments, a machine-learning based system for mental health disorder identification and monitoring is disclosed. The system includes one or more processors configured to interact with a plurality of wearable medical sensors (WMSs). The processors are configured to receive physiological data from the WMSs. The processors are further configured to train at least one neural network based on raw physiological data augmented with synthetic data and subjected to a grow-and-prune paradigm to generate at least one mental health disorder inference model. The processors are also configured to output a mental health disorder-based decision by inputting the received physiological data into the generated mental health disorder inference model.

According to various embodiments, a machine-learning based method for mental health disorder identification and monitoring utilizing one or more processors configured to interact with a plurality of wearable medical sensors (WMSs). The method includes receiving physiological data from the WMSs. The method further includes training at least one neural network based on raw physiological data augmented with synthetic data and subjected to a grow-and-prune paradigm to generate at least one mental health disorder inference model. The method also includes outputting a mental health disorder-based decision by inputting the received physiological data into the generated mental health disorder inference model.

According to various embodiments, a non-transitory computer-readable medium having stored thereon a computer program for execution by a processor configured to perform a machine-learning based method for mental health disorder identification and monitoring is disclosed. The method includes receiving physiological data from a plurality of WMSs. The method further includes training at least one neural network based on raw physiological data augmented with synthetic data and subjected to a grow-and-prune paradigm to generate at least one mental health disorder inference model. The method also includes outputting a mental health disorder-based decision by inputting the received physiological data into the generated mental health disorder inference model.

Various other features and advantages will be made apparent from the following detailed description and the drawings.

### BRIEF DESCRIPTION OF THE DRAWINGS

In order for the advantages of the invention to be readily understood, a more particular description of the invention briefly described above will be rendered by reference to specific embodiments that are illustrated in the appended drawings. Understanding that these drawings depict only exemplary embodiments of the invention and are not, therefore, to be considered to be limiting its scope, the invention will be described and explained with additional specificity and detail through the use of the accompanying drawings, in which:

FIG. 1 depicts an MHDeep mental health disorder detection system according to an embodiment of the present invention;

FIG. 2 depicts a schematic diagram of the MHDeep framework according to an embodiment of the present invention;

FIG. 3 depicts a smartwatch and smartphone used in the data collection process according to an embodiment of the present invention;

FIG. 4 depicts a table of data types collected in the MHDeep framework according to an embodiment of the present invention;

FIG. 5 depicts a table of details of various datasets for major depressive disorder according to an embodiment of the present invention;

FIG. 6(a) depicts an architecture of MHDeep NNs for healthy vs major depressive disorder and healthy vs schizoaffective disorder according to an embodiment of the present invention;

FIG. 6(b) depicts an architecture of MHDeep NNs for healthy vs bipolar disorder according to an embodiment of the present invention;

FIG. 7 depicts a schematic diagram of the MHDeep synthetic data generation process according to an embodiment of the present invention;

FIG. 8 depicts a grow-and-prune synthesis algorithm according to an embodiment of the present invention;

FIG. 9 depicts a table of test accuracy, FPR, FNR, and F1 score (all in %) for top data categories for classification between healthy and schizoaffective disorder data instances according to an embodiment of the present invention;

FIG. 10 depicts a table of test accuracy, FPR, FNR, and F1 score (all in %) for top data categories for classification between healthy and major depressive disorder data instances according to an embodiment of the present invention;

FIG. 11 depicts a table of test accuracy, FPR, FNR, and F1 score (all in %) for top data categories for classification between healthy and bipolar disorder data instances according to an embodiment of the present invention;

FIG. 12 depicts a table of a comparison with previous machine learning models on a first data partition according to an embodiment of the present invention;

FIG. 13 depicts a table of an impact of different training methods on the performance of the model according to an embodiment of the present invention;

FIG. 14(a) depicts patient-level test accuracy vs duration of data needed for classification between healthy and schizoaffective disorder individuals according to an embodiment of the present invention;

FIG. 14(b) depicts patient-level test accuracy vs duration of data needed for classification between healthy and major depressive disorder individuals according to an embodiment of the present invention;

FIG. 14(c) depicts patient-level test accuracy vs duration of data needed for classification between healthy and bipolar disorder individuals according to an embodiment of the present invention;

FIG. 15 depicts a table of minimum inference data duration (in minutes) needed to reach saturation patient-level accuracy (in %) for each classification task according to an embodiment of the present invention; and

FIG. 16 depicts a table of a comparison with other works according to an embodiment of the present invention.

### DETAILED DESCRIPTION OF THE INVENTION

Mental health problems impact the quality of life of millions of people around the world. However, identifying mental health disorders is a challenging problem that often relies on self-reporting by patients about their behavioral patterns and social interactions. Therefore, there is a need for new strategies for identification and daily monitoring of mental health conditions.

The recent introduction of body-area networks including a plethora of accurate sensors embedded in smartwatches and smartphones and edge-compatible neural networks (NNs) points towards a possible solution. Such wearable medical sensors (WMSs) enable continuous monitoring of physiological signals in a passive and non-invasive manner. However, disease diagnosis based on WMSs and NNs, and their deployment on edge devices, such as smartphones, remains a challenging problem. These challenges stem from the difficulty of feature engineering and knowledge distillation from the raw sensor data, as well as the computational and memory constraints of battery-operated edge devices.

To this end, generally disclosed herein are embodiments for a mental health detection framework called MHDeep that utilizes WMSs and efficient NN models to identify important mental health disorders in users including but not limited to schizoaffective, major depressive, and bipolar. MHDeep relies on physiological data collected using various WMSs, which can be used to continuously monitor the physiological signals of the wearer to enable constant tracking of the health conditions of the user. MHDeep uses eight different categories of data obtained from sensors integrated in a smartwatch and smartphone. These categories include various physiological signals and additional information on motion patterns and environmental variables related to the wearer.

For training purposes, the collected physiological data are processed to obtain a comprehensive dataset. MHDeep combines data from WMSs with the inference capabilities of NNs to directly extract mental health condition from the physiological signals. These inferences can be communicated to a health server that is accessible to the physician. This has the potential to enhance the ability of the physician to intervene quickly when mental health conditions deteriorate.

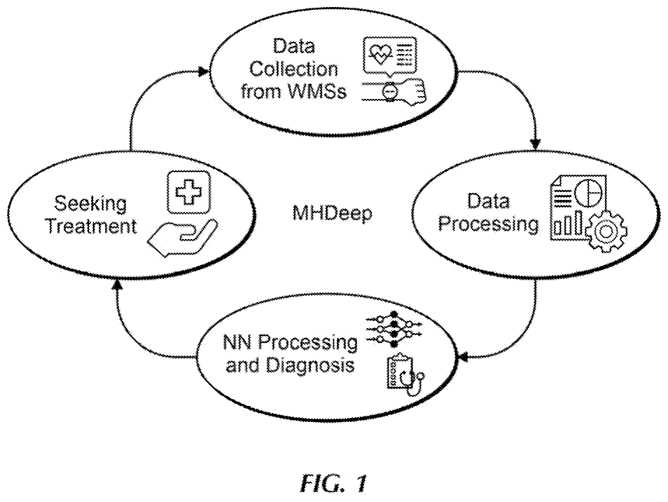

The difficulty of data collection and labeling limits the amount of available data so reducing the cost of this process is of great importance. MHDeep eliminates the need for manual feature engineering by directly operating on the data streams obtained from participants. Since the amount of data is limited, MHDeep uses a synthetic data generation module to augment real data with synthetic data drawn from the same probability distribution. The synthetic dataset is used to pre-train the weights of the NN models, thus imposing a prior on the weights. A grow-and-prune NN synthesis approach is used to learn both architecture and weights during the training process. This trains accurate and computationally efficient NN models that can detect the mental health condition of the user. FIG. 1 depicts a general overview of the MHDeep mental health disorder detection framework.

Three different data partitions to evaluate the MHDeep models trained with data collected from 74 individuals. Two types of evaluations are conducted: at the data instance level and at the patient level. MHDeep achieves an average test accuracy across the three data partitions of 90.4%, 87.3%, and 82.4%, respectively, for classifications between healthy and schizoaffective disorder instances, healthy and major depressive disorder instances, and healthy and bipolar disorder instances. At the patient level, MHDeep NN models achieve an accuracy of 100%, 100%, and 90.0% for the three mental health disorders, respectively, based on inference that uses 40, 16, and 22 minutes of data from each patient.

### System Overview

This section provides information on various mental health disorders and how they affect patient lives. Next, various methods for identifying mental health conditions based on machine learning and synthesizing efficient NN models are described. Finally, WMSs and their applications to various disease identification frameworks are described.

Mental Health Disorders and their Impact

Mental health conditions can affect a patient's thinking, feeling, and behavior. They may have a deep impact on the daily life of the person and affect their ability to adequately perform in society. There are hundreds of different mental illnesses so three major mental health illnesses targeted herein are bipolar, major depressive, and schizoaffective. However, that is not intended to be limiting.

Bipolar disorder can cause a dramatic shift in a person's mood, energy, and behavior. It is characterized by experiences of alternating episodes of manic and depressed states. Major depressive disorder may present different symptoms like loss of interest, sleep disturbance, change in appetite, and feeling of fatigue. Schizoaffective disorder is characterized by various symptoms of schizophrenia such as episodes of hallucinations and delusions. It may also present other symptoms such as disorganized thinking, depressed mood, and manic behavior.

Apart from various conditions that these mental health disorders may cause, stereotypes related to mental health seem to still be widely prevalent in society, not just among lay people but even well-trained professionals as well. These stereotypes often lead to social and employment discrimination and poor treatment of physical health problems.

### Deep Learning for Mental Health

Deep learning has been recently used to better understand and detect mental health problems. Deep learning approaches have been applied to various types of data: mainly clinical data and social media usage data. The three types of clinical data used have been neuroimage data, EEG data, and EHR data.

Several studies have demonstrated the effectiveness of neuroimages in detecting neuropsychiatric disorders. Two types of neuroimage data used in such works are functional magnetic resonance imaging (fMRI) and structural magnetic resonance imaging (sMRI). fMRI measures brain activity by monitoring blood oxygenation and flow in response to neural activity. sMRI examines the anatomy and pathology of the brain. Deep belief networks have been used to detect the presence of attention-deficit hyperactivity disorder (ADHD) using fMRI and sMRI data. These data types have also been used to detect schizophrenia. Depression has been detected using time-series fMRI data using convolutional neural networks (CNNs) and autoencoders. EEG is another source of data for studying brain disorders. For example, CNN-based feature extraction from EEG data has been used to detect depression. EHR is a collection of patient-centered records and includes both structural data such as laboratory reports and unstructured data such as clinical and discharge notes. Since EHR is a collection of longitudinal records, recurrent neural networks (RNNs) have been used to distill information from them. RNN architectures have been used to predict future outcomes of depressive episodes. Unstructured clinical notes have also been analyzed with deep learning-based models to detect depression. Social media usage data have also proved their usefulness in identifying psychiatric illnesses. Facebook messages and patterns of sharing images were investigated on social media to distinguish among healthy individuals, individuals with a schizophrenia spectrum disorder, and individuals with mood disorders. Other works have used NN models with textual data and image data shared on social media platforms to detect stress, depression, and risk of suicide.

MHDeep relies on generating synthetic data from the same distribution as the real data. Several compact and accurate dynamical models exist that capture characteristics of the system under study. Synthetic data can be generated based on these mathematical models. A multivariate fractal model has been proposed to capture long-range memory and spatial dependencies that exist in biological processes and show the benefits of this approach within the context of the brain-machine-body interface. A mathematical strategy for constructing models of complex nonlinear dynamics has also been proposed. Another proposal involves a polynomial-time algorithm to obtain a suboptimal solution with optimality guarantees for the NP-hard problem of determining the minimum number of sensors to enable recovery of global data dynamics. Although MHDeep uses deep learning for mental health disorder identification, it is worth mentioning that deep learning has also been used in other use cases such as in vaccine discovery for SARS-COV-2.

### Efficient Neural Network Synthesis

Next are summaries of the main synthesis approaches for obtaining compact deep neural network (DNN) models. Previous synthesis methods have been based on the use of efficient building blocks. For example, one synthesis method leverages inverted residual blocks to reduce model size and computations significantly. Another uses shift-based operations rather than convolution layers to significantly reduce computational costs of the model. The main drawback of such approaches is the need for considerable design insight and trial-and-error process to design such efficient building blocks.

Network compression is another approach for the design of efficient models as it removes the need for design insights. Network pruning is a widely used method that eliminates weights or filters that do not enhance model performance. The effectiveness of pruning in removing redundancy in CNNs and multilayer-perceptron architectures has been shown. Grow-and-prune NN synthesis uses network growth followed by network pruning in an iterative process to improve model performance while ensuring its compactness.

Another recent approach relies on the use of reinforcement learning (RL) to search for DNN architectures in an automated flow, known as neural architecture search (NAS). NAS generally uses a controller, e.g., an RNN, to iteratively generate candidate architectures in the search process. The RL controller is improved based on candidate performance. As an example, an RL-based approach has been used to develop efficient DNNs for mobile platforms. However, the downside of the RL-based NAS approach is that it is computationally intensive. Further improving the NAS approach, a Gumbel softmax function has been used to optimize weights and connections using a single objective function. Further, evolutionary algorithms have been used to generate optimized and increasingly complex architectures over multiple generations. Combining efficient evolutionary search algorithms with various performance predictors, e.g., for accuracy, energy, and latency, is another approach for synthesizing accurate yet compact CNNs and DNNs.

### Wearable Medical Sensors

Due to recent developments in low-power sensor design and efficient wireless communication, battery-powered WMSs are becoming ubiquitous. More than 123 million WMSs were sold worldwide in 2018. This number is projected to grow to 1 billion by the end of 2022. WMSs can track different aspects of human health including heart rate, body/skin temperature, respiration rate, blood pressure, EEG, electrocardiogram (ECG), and Galvanic skin response (GSR). Furthermore, the number of physiological signals that can be measured using WMSs keeps growing every year.

WMSs have begun to be used in many smart healthcare applications. A sensor network exists that collects vital health signs and transmits them to the healthcare provider. Further, a WMS-based body-area network (BAN) realizes an end-to-end mobile health monitoring platform. WMSs have also been used for pervasive identification of Type-I and Type-II diabetes as well as for quick detection of SARS-COV-2/COVID-19.

For data collection in MHDeep, an Empatica E4 smartwatch was used to record a subset of patient's physiological signals. It is a wearable wireless device configured for comfortable, continuous, and real-time data acquisition. A smartphone was also used to simultaneously record signals related to motion information and environmental variables. Since the NNs developed for diagnosing various mental health conditions can reside on the smartphone, use of a smartwatch/smartphone-based BAN can enable accurate, yet convenient, disease diagnosis and continuous healthcare monitoring. However, it should be noted the particular WMSs used herein (such as smartwatches and smartphones) are not intended to be limiting and only provided for exemplary purposes.

### Methodology

Herein are various parts of the MHDeep framework. First is an overview of the disclosed approach. Then, the data collection and preparation process, synthetic data generation, and grow-and-prune NN synthesis are described.

### MHDeep Framework

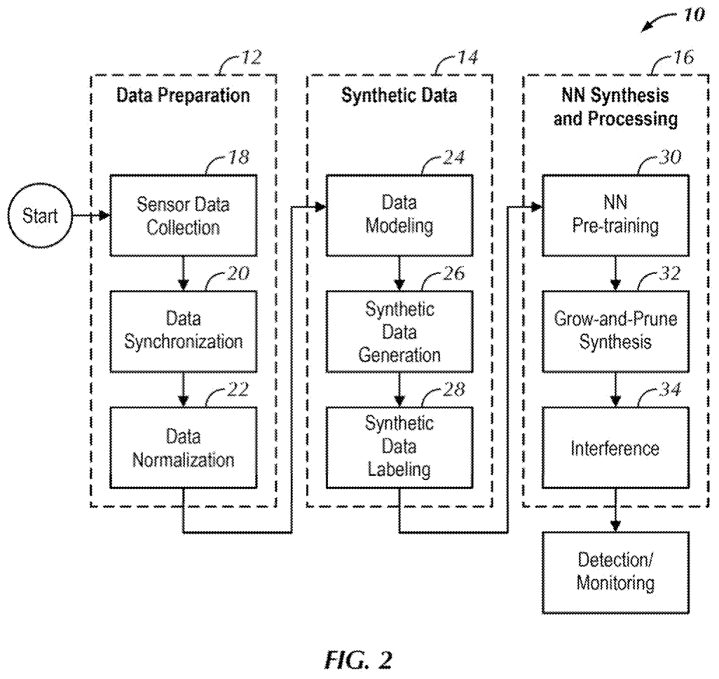

The MHDeep framework 10 is illustrated in FIG. 2, with three general steps: data preparation 12, synthetic data preparation 14, and NN synthesis and processing 16. Data preparation 12 starts with sensor data collection 18. Input data are derived from physiological signals collected using various WMSs in a noninvasive, passive, and efficient manner, such as from a smartwatch and/or smartphone. The list of collected data streams include but are not limited to GSR, skin temperature (ST), inter-beat interval (IBI), and 3-way acceleration (tri-axial accelerometer) from the smartwatch. In addition, some information related to the motion patterns of the user and ambient information are collected using smartphone sensors. This includes but is not limited to ambient temperature, gravity, acceleration, and angular velocity.

After sensor data collection 18, the collected signals are synchronized, aggregated, and merged into a comprehensive data input for subsequent analysis 20. To enhance the accuracy of subsequent analysis and improve noise tolerance, the data is normalized 22.

When the size of the training dataset is small, it can be useful to prepare a synthetic dataset from the same probability distribution as the real training dataset. Synthetic data preparation 14 includes data modeling 24 and leveraging a Gaussian mixture model (GMM)-based density estimation to generate the synthetic data 26. The synthetic data is then labeled 28.

NN synthesis and processing 16 includes a NN pre-training step 30. Then, MHDeep 10 uses grow-and-prune NN synthesis 32 to generate inference models 34 that are both accurate and computationally efficient. MHDeep 10 generates NN architectures that are efficient enough to be deployed on the edge devices such as smartphones or smartwatches.

### Data Preparation 12

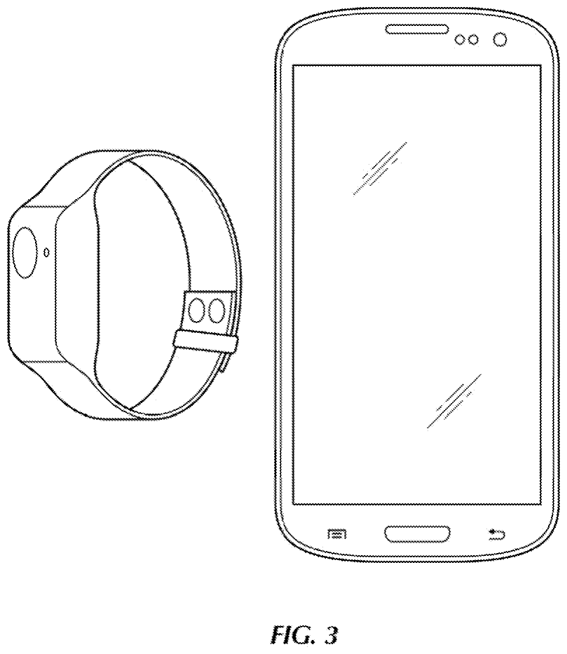

For the exemplary embodiment disclosed herein, at sensor data collection 18, WMS data was collected from a total of 74 adult participants. The participants were categorized by medical professionals into the following four categories: 25 healthy participants (no mental health disorder), 23 participants with bipolar disorder, 10 participants with major depressive disorder, and 16 participants with schizoaffective disorder. The physiological signals of the participants were captured by a smartwatch and smartphone, here the Empatica E4 smartwatch and Samsung Galaxy S4 smartphone, respectively, as shown in FIG. 3.

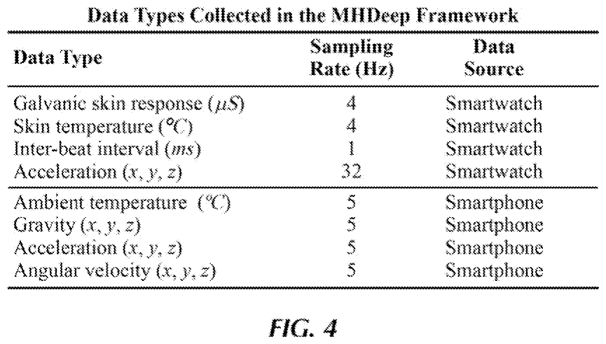

All the data types collected are summarized in the table in FIG. 4. The physiological signals are derived from WMSs embedded in the smartwatch. Here, they include GSR that measures sympathetic nervous system arousal, IBI that indicates the heart rate, ST that provides skin temperature readings, and 3-axis accelerometer (Acc-W) that measures acceleration in the G, H, and I directions.

The information collected from these sensors is useful for detecting various mental disorders. For example, the electrodermal response can be used as a feature to detect the patients affected by depression disorder, or to detect different mood disorders. Bipolar disorder is also associated with cardiac autonomic dysregulation that has an impact on IBI.

In addition to the physiological signals, ambient and motion information is also captured using sensors in the smartphone. Here, these include ambient temperature (Temp), gravity (Grav), acceleration (Acc-P), and angular velocity (Vel). The motion and ambient information may also be informative in detecting the mental state of the user. For example, it has been shown that motor activities of schizophrenic and depressed patients are significantly reduced.

It is worth mentioning that the acceleration sensors in the smartphone and smartwatch have different sampling rates and capture different motion information. Initially, data were obtained from an extensive set of sensors embedded in both the smartwatch and smartphone. This set included sensors such as blood volume pulse in the Empatica E4 smartwatch and sensors such as ambient pressure, light, humidity, magnetism, and gyroscope in the smartwatch. The mean value and standard deviation of collected data was analyzed from each sensor for the four patient cohorts (healthy and three disorders). The final set of eight sensors was identified as being the most informative in terms of distinguishing among these four cohorts.

The data collection setup includes placing the Empatica E4 smartwatch on the wrist of the participant's non-dominant hand and placing the Samsung Galaxy S4 smartphone in the opposite front pocket. The same orientation for the phone is maintained for all participants. Data collection lasts around 1.5 hours, during which time the participant is allowed to freely move around in the room with their on-body devices. During this time, the smartwatch and smartphone continuously record and store physiological signals and ambient/motion information. At the end of the data collection period, the smartwatch is removed from the patient's wrist and the smartphone is removed from the pocket. A data repository, such as the cloud based Empatica E4 Connect portal and a private Android application, is used for smartwatch and smartphone data retrieval, respectively. All of the recorded data are timestamped at the time of sampling.

Next, the dataset is preprocessed for use in NN training. First, the smartwatch and smartphone data streams are synchronized for each participant 20. This is necessary since the WMS data streams may vary in their start times and frequencies. Then, for data normalization 22, the data are divided for each participant into 15-second windows. This window size was chosen based on experiments with the validation set, as discussed later, though it is not intended to be limiting. Each 15-second window of the combined smartwatch/smartphone data constitutes one data instance. There is no time overlap between data instances. To obtain each data instance, the data are flattened and concatenated within the same time window from both the smartwatch and smartphone. This results in a feature space of dimension 2325. The smartwatch (smartphone) contributes 1575 (750) features. All the smartphone sensors have a sampling rate of 5 Hz. In addition, the smartwatch sensors include one data stream at 32 Hz, two data streams at 4 Hz, and one data stream at 1 Hz. However, these frequencies are not intended to be limiting.

Since the participants are in a room during the data collection process, they do not enjoy a wide range of motions, and hence the higher sampling rates for the smartphone sensors are not needed. In addition, the Empatica E4 used for data collection is a medical-grade smartwatch that is configured to capture various physiological signals with their optimal sampling rates. Although collecting data from more sensors at higher sampling rates may provide more information, unnecessarily high sampling rates can lead to a decrease in the battery life of the device. In addition, by targeting a window of 15 seconds for each data instance, the low sampling frequency of some of the sensors can be remedied by considering multiple sensor readings in each data instance.

For each classification task, since the number of individuals in each of the four categories (healthy and three disorders) is small, three different data partitions are created for evaluation. Circular shifts are used on a list of numbers denoting patients in each category to create these three data partitions. The value of the stride used for the circular shift is equal to the number of test individuals in each group (as explained further below). The data instances extracted from the individuals in each of the four groups (healthy, schizoaffective, depressive, and bipolar) were divided into three sets: training, validation, and test. To evaluate the models on different unseen patients, data instances included in the training, validation, and test sets came from different individuals, i.e., no individual contributed data to more than one of these sets. Among the healthy participants, for each of the three data partitions, data instances from 15 individuals (60% of the healthy participants) are selected for the training set, from 5 individuals (20% of the healthy participants) for the validation set, and from the remaining 5 individuals (20% of the healthy participants) for the test set. For individuals with bipolar disorder, the training, validation, and test sets contain data instances from 13, 5, and 5 participants, respectively. Among the participants who had major depressive disorder, data instances from 6 participants are selected for the training set and from 2 participants each for the validation and test sets. For individuals with schizoaffective disorder, the training, validation, and test sets include data instances from 10, 3, and 3 participants, respectively.

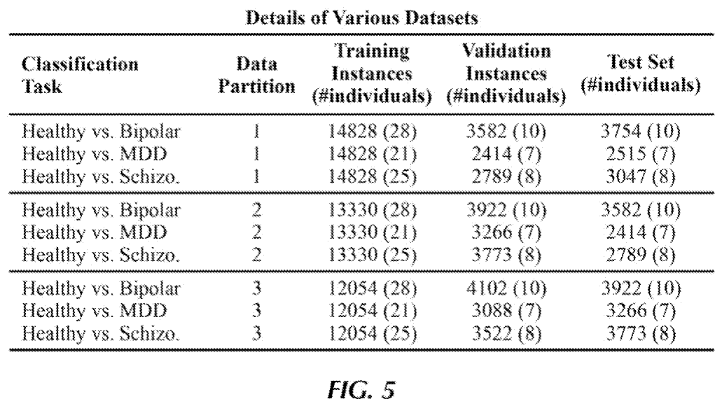

The final dataset for each binary classification task (healthy vs. the mental health disorder) is created by combining the training, validation, and test sets of the two classes involved in that task. A synthetic minority upscaling technique (referred to as SMOTE) is used to up-sample data instances from the minority class. SMOTE creates new samples from the minority class. It first selects samples that are close to each other in the feature space. By connecting these samples together, SMOTE generates new samples at a point along this connecting line. Up-sampling is only applied to the training set. The table in FIG. 5 shows the number of instances for each of the classification tasks for all three data partitions.

### MHDeep NN Synthesis

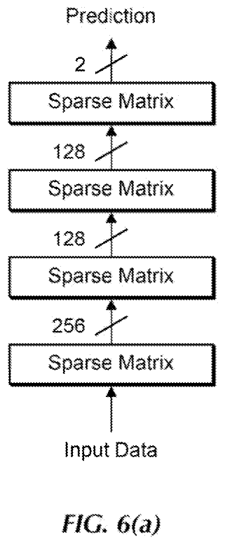

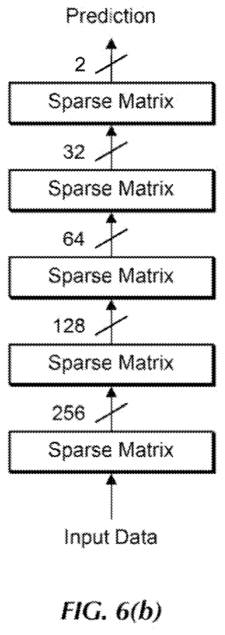

FIGS. 6(a)-(b) show the NN architectures used in the MHDeep framework. The architectures receive the input data at the bottom and make their diagnostic decisions at the top. Shown in FIG. 6(a), for the healthy vs. major depressive disorder and healthy vs. schizoaffective disorder binary classification tasks, the NN architecture has four layers with a width of 256, 128, 128, and 2, respectively. Shown in FIG. 6(b), for the healthy vs. bipolar disorder binary classification task, an NN architecture with five layers is used with a width of 256, 128, 64, 32, and 2, respectively. These architectures were selected by verifying the performance of various NNs (with different numbers of layers and number of neurons per layer) on the validation set and picking the best-performing one. As such, they are not intended to be limiting. These architectures are initially fully connected.

Three sequential steps are then applied: (i) synthetic data preparation 14 to mimic the distribution of the real training data, (ii) pre-training of the NN architectures with the synthetic data 30, and (iii) grow-and-prune NN synthesis 32 to reduce the redundancy of the model while improving its performance. Each step is discussed below in more detail.

### Synthetic Data Preparation 14:

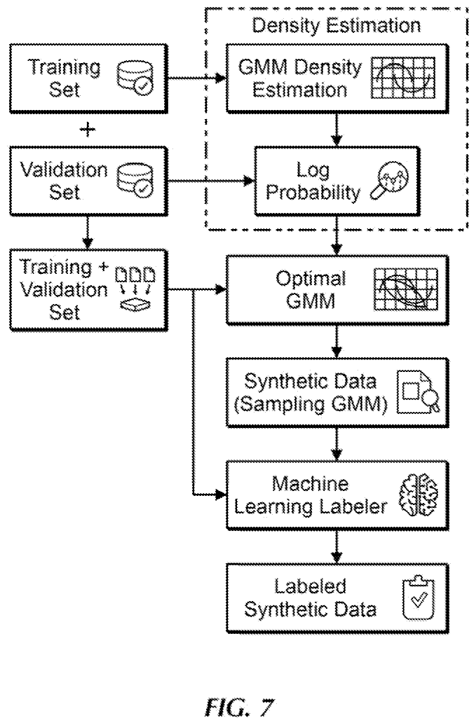

Here, data is modeled 24 and then synthetic data is generated 26 that mimics the probability distribution of the real training dataset. FIG. 7 illustrates the synthetic data generation process. An approach referred to as TUTOR is utilized to alleviate the need for large datasets to train NN architectures. First, GMM is used to estimate the density of the training dataset. The number of mixtures is optimized in the GMM by monitoring the likelihood of validation data. The number of components that maximizes the following criterion is chosen:

$${N}^{*} = {arg}_{N} max ( G M {M}_{N} ( x ) \cdot score ( {X}_{validation} ) ) \qquad ( 1 )$$

The log probability of the validation data instances (the criterion that is being maximized) is used to compare various GMM models with different number of mixtures. As a result, the optimal value for the number of mixtures (the number of mixtures that leads to the maximum value for the criterion mentioned above) can be determined.

The optimal GMM model with N* mixtures is trained on the combination of the training and validation datasets. By sampling this model, synthetic data is able to be generated:

$${X}^{*} = G M {M}_{{N}^{*}} ( {X}_{t o t a l} ) \cdot sample ( ) \qquad ( 2 )$$

GMM provides a probability density function from which samples are drawn to generate the synthetic data.

For the exemplary embodiment, 100,000 samples are generated as synthetic data. The final step is labeling of the synthetic dataset 28. A machine learning model is used for this purpose. Various models, e.g., the support vector machine and random forest models based on different splitting criteria (such as Gini index and entropy), and different depth limits on the decision trees, on the validation set. The model with the highest accuracy is used to label the synthetic data. Note that since synthetic data are only used to pre-train an NN (with subsequent training with real data), the accuracy of the support vector machine or random forest model is not a critical factor. Therefore, the particular machine learning model used here is not intended to be limiting.

### NN Pre-Training 30:

In this step, the labeled synthetic data is used to obtain a prior on the weights of the NN architecture by pretraining them. The intuition behind this step is that pre-training the NN provides a suitable inductive bias to the parameters of the NN. As a result, the final training stage can be commenced with a better weight initialization. Therefore, it alleviates the need for large training datasets. With this methodology, models are obtained that are more accurate compared to both typical machine learning models used for labeling and an NN model trained only on the real dataset.

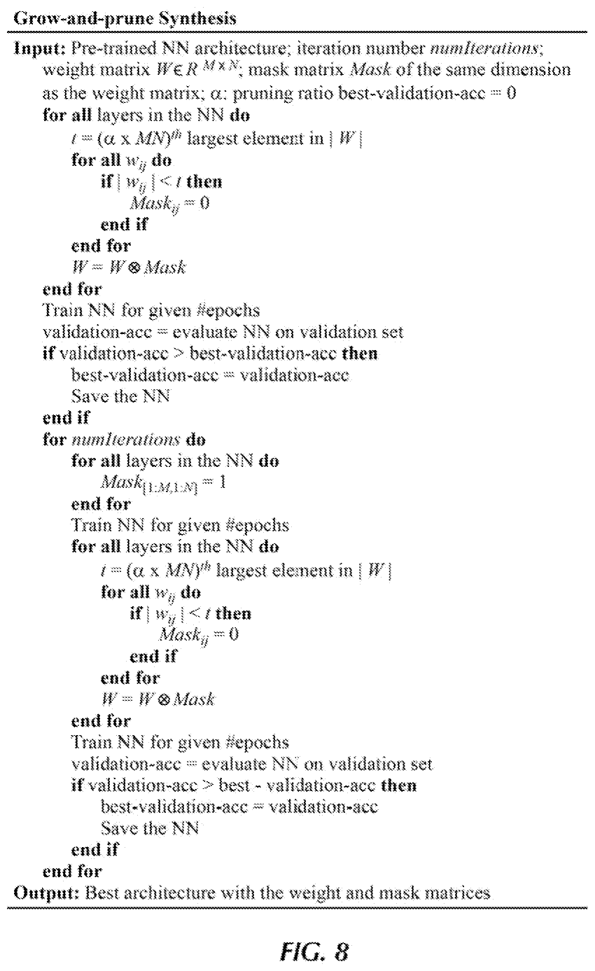

Grow-and-prune NN synthesis 32: MHDeep uses a grow-and-prune NN synthesis paradigm to train the models. The algorithm in FIG. 8 summarizes this process. It uses a mask-based approach. For each weight matrix, there is an associated binary mask of the same size that is used to disregard dormant connections in the architecture. It applies magnitude-based pruning and full growth to fully connected NNs iteratively. For magnitude-based pruning, a hyperparameter a is used to depict the pruning ratio. A connection is pruned if and only if its weight is in the lowest α*100 percent of the weights in its associated layer. Finally, for the pruned connections, the weight and its binary mask are both set to 0. Since connection pruning is an iterative process, the network is retrained to recover its performance after each pruning iteration. The network is then grown to restore all its connections. In the exemplary embodiment, after each architecture-changing operation, the NN is trained for 20 epochs. In addition, the number of iterations is set to 5. The model is evaluated on the validation set after each epoch and the learned weights and masks of the pruned model with the highest validation accuracy are recorded.

### MHDeep Inference Process 34

The trained NN models can be used for identification or daily monitoring of the mental state of the user based on a collection of physiological signals and ambient information during the day. The collected data streams are processed using the steps described earlier on data collection and preparation. The processed data is fed to the MHDeep NN models that predict the mental health condition of the user. When the model predicts the presence of the mental health disorder, this information can be sent to a physician for early treatment.

### Exemplary Implementation Details

This section gives an overview of the hardware and software packages used in the exemplary implementation of the MHDeep framework. It is not intended to be limiting and similar hardware and software packages can be used in alternative embodiments. The data processing and preparation parts of the MHDeep framework are implemented in Python and the NN synthesis part in PyTorch. The Nvidia Tesla P100 data center accelerator is used for NN training and evaluation. The cuDNN library is used to accelerate GPU processing. For training, a stochastic gradient descent (SGD) optimizer is used, with a learning rate of 5e-4 and a batch size of 256. 100,000 synthetic data instances are used to pre-train the network architecture. In the grow-and-prune synthesis phase, the network is trained for 20 epochs each time the architecture changes. An SGD optimizer is used, with an initialized learning rate of 1e-4 that is halved in each succeeding iteration. Network-changing operations are applied over five iterations.

### Evaluation

This section analyzes the performance of MHDeep NN models for diagnosing three mental health disorders. This entails three binary classifications: (i) schizoaffective disorder vs. healthy individuals, (ii) major depressive disorder vs. healthy individuals, and (iii) bipolar disorder vs. healthy individuals. For each classification task, three different data partitions are used, each partition with data instances obtained from different individuals in the training, validation, and test sets.

The MHDeep NN models are evaluated with four different metrics: test accuracy, false positive rate (FPR), false negative rate (FNR), and F1 score. Accuracy measures overall classification performance. It is simply the ratio of all the correct predictions on the test data instances and the total number of such instances. FPR and FNR measure how often healthy individuals are declared to have the corresponding mental health condition and vice versa, respectively. In addition to these four metrics, also reported are the TPR (sensitivity) and the TNR (specificity) values that are equal to 1 minus the FNR and FPR values, respectively.

Two different performance evaluations are conducted: at the data instance level and the patient level. First, the performance of the MHDeep NN models in detecting each of the three mental health disorders are reported at the data instance level. Next, the accuracy of the models in detecting mental health disorders at the patient level are evaluated.

### MHDeep Performance Evaluation at the Data Instance Level

The performance of the three binary classifiers are first analyzed. NN models are trained on features obtained from subsets of the eight data categories presented in the table in FIG. 4. All of the subsets of the eight data categories are analyzed and the results are reported for the top models. Since there are eight data categories, there are 256 subsets, with one being the null subset. The remaining 255 subsets are evaluated. This helps distinguish the impact of each data category and to find the most effective combination of categories for each classification task. Next, the best-performing data categories are highlighted for each of the three classification tasks. The performance of MHDeep DNNs is then compared with other machine learning models. An ablation study that shows the impact of each step of MHDeep DNN training is also described.

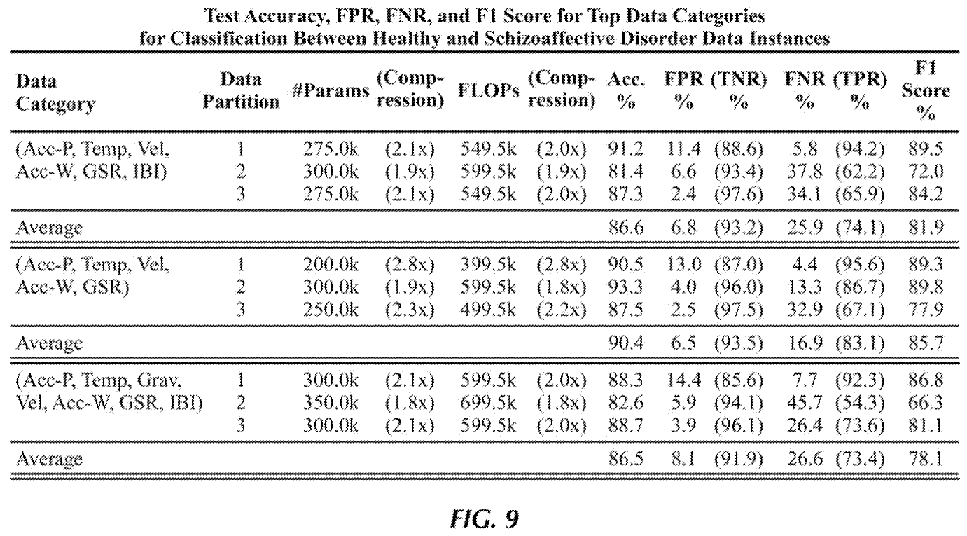

The table in FIG. 9 shows the results of classification between healthy and schizoaffective data instances. The best data category subset, in this case, achieves an average test accuracy of 90.4%. Test accuracy, FPR, FNR, TPR, TNR, and F1 score are also reported for each of the three data partitions. The model reaches the highest test accuracy of 93.3% on the second data partition. Furthermore, for the healthy instances, the top model achieves a low average FPR of 6.5%, demonstrating its effectiveness in avoiding false alarms. For the schizoaffective instances, the model achieves an average FNR of 16.9%, indicating reasonable effectiveness in raising alarms when schizoaffective disorder does occur. The number of parameters (#params) and floating-point operations (FLOPs) required for each model are reported. #params and FLOPs of the models are also compared with those of the fully-connected baselines. As can be seen, using the grow-and-prune NN synthesis approach enables reduction of both #params and FLOPs, leading to a reduction in memory and computational requirements.

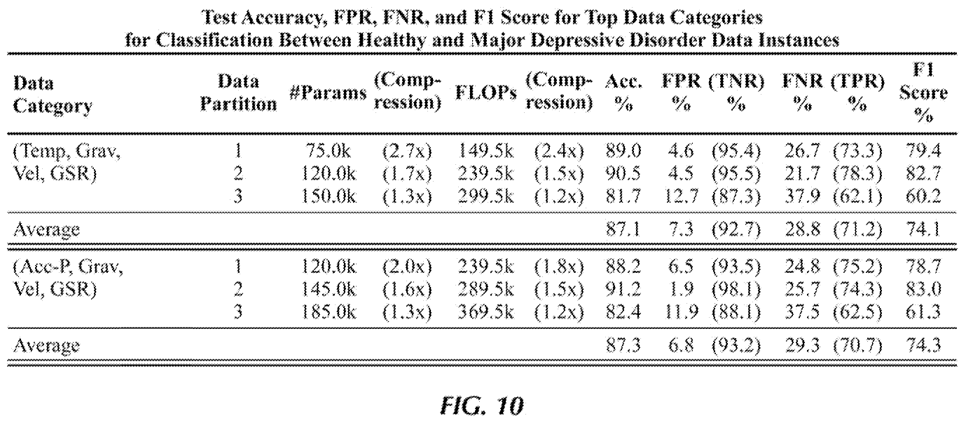

The results for classification between healthy and major depressive disorder instances are presented in the table in FIG. 10. The data category subset with the best performance achieves an average test accuracy of 87.3%. This model achieves the highest accuracy of 91.2% on the second data partition. It achieves an average FPR (FNR) of 6.8% (29.3%). The table in FIG. 11 presents the results for classification between healthy and bipolar disorder instances. In this case, the model trained on the best data category subset achieves an average test accuracy of 82.4%, with an FPR (FNR) of 16.7% (20.7%).

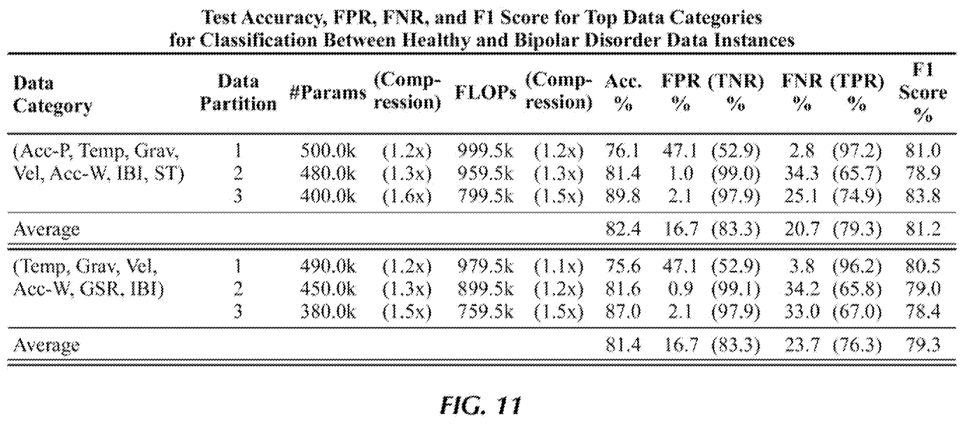

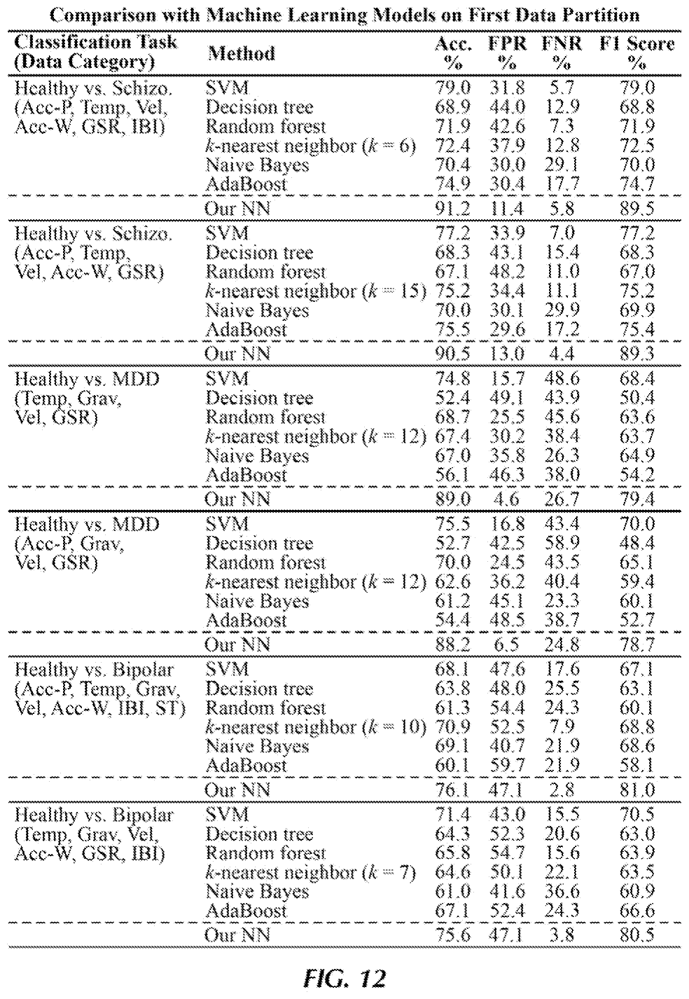

Next, the performance of MHDeep is compared with other machine learning methods. To this end, the results on the first data partition are presented in the table in FIG. 12. The results are reported on two data categories for each classification task. As can be seen, in all cases, MHDeep outperforms other machine learning models. This points to the difficulty of distilling information from raw data using other machine learning models and highlights the superior performance here in identifying various mental illnesses. It is worth mentioning that similar results were achieved on the second and third data partitions.

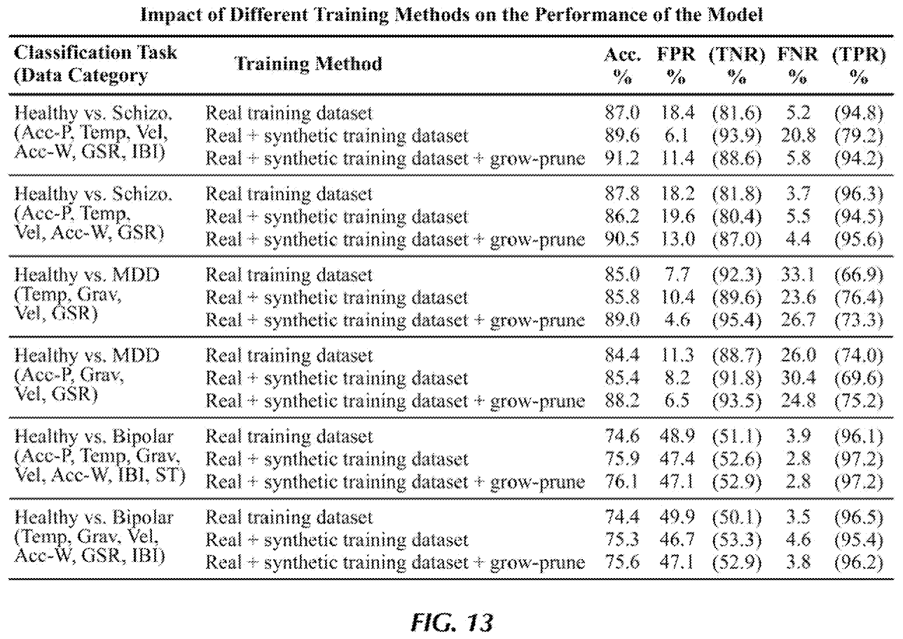

An ablation study was also performed to analyze the impact of three training methods on the final performance, including training only on real data, pre-training with synthetic data, and grow-and-prune synthesis. To this end, performance results on the first data partition are presented in the table in FIG. 13. For each classification task, the performance of two data categories is reported. It can be seen that using the synthetic dataset to pre-train the weights of the model helps improve performance in most cases, thanks to a better initialization point for the final training process. In addition, the application of grow-and-prune synthesis not only results in more compact models (as shown in FIGS. 9-11), but also improves the performance of the models as well. It is worth mentioning that similar results were obtained on the other two data partitions.

### MHDeep Performance Evaluation at the Patient Level

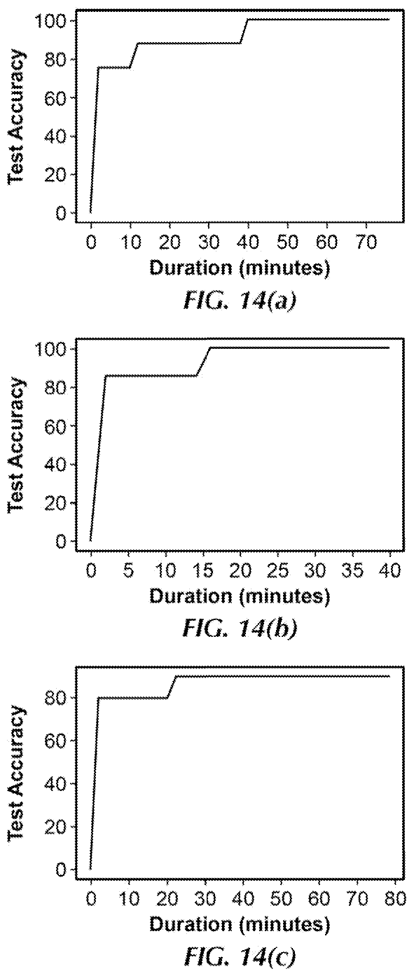

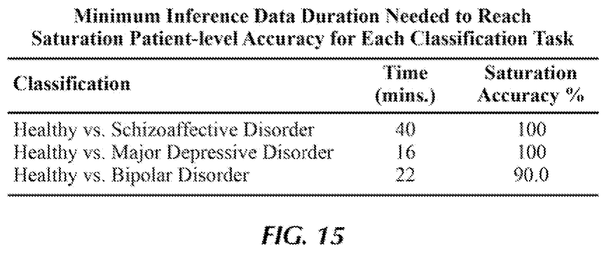

Next, patient-level diagnostic test accuracy is shown. The most accurate model from among the models discussed above is used for each classification task. FIGS. 14(a)-(c) show the results. In these graphs, patient-level test accuracy vs. the duration of data needed for inference is plotted. Prediction is performed for each patient by simply taking the majority of the predicted labels for each data instance in the given data duration. As discussed, each data instance is composed of a 15-second window of the sensor data. The data duration size is stepped up by 2 minutes each time. Thus, eight data instances are added in each 2-minute window. By predicting the label for each participant, the final test accuracy is defined as the ratio of the participants that are correctly diagnosed over the number of participants in the test set. As can be seen, the models reach 100% test accuracy after a certain point for distinguishing healthy individuals from those with schizoaffective and major depressive disorders. In addition, the best model for classification between healthy and bipolar disorder individuals reaches 90.0% patient-level accuracy. The table in FIG. 15 shows the minimum data duration needed to reach saturation accuracy. The durations are 40, 16, and 22 minutes for healthy vs. schizoaffective disorder, healthy vs. major depressive disorder, and healthy vs. bipolar disorder classifications, respectively.

Although MHDeep DNNs were evaluated on the same platform used for training, these models can also be deployed in a smartphone application (app) to identify mental disorders. In this case, through the MHDeep app, the user can be instructed to wear a smartwatch (such as the Empatica E4 smartwatch) and correctly place a smartphone before data collection commences. After data collection, the MHDeep preprocessing pipeline may be used to normalize the data using the minimum and maximum values used in the training process and divide the data into data instances with a 15-second window. Finally, through DNN processing of various mental disorders, the average prediction probabilities can be obtained. The three mental disorders can be identified based on a threshold.

The MHDeep app would need a limited amount of battery energy. It is estimated between 0.5 to 1 Watt of battery power is needed for the application. Assuming 60 minutes of data are needed for inference in the worst case (as explained above, at most 40 minutes of data was needed) and since the smartphone battery works at 3.8V, this translates to 131-263 mAh energy consumption. For a smartphone, such as Samsung Galaxy S8+ with a battery of 3500 mAh capacity, this results in 3.7% to 7.5% battery consumption for the app.

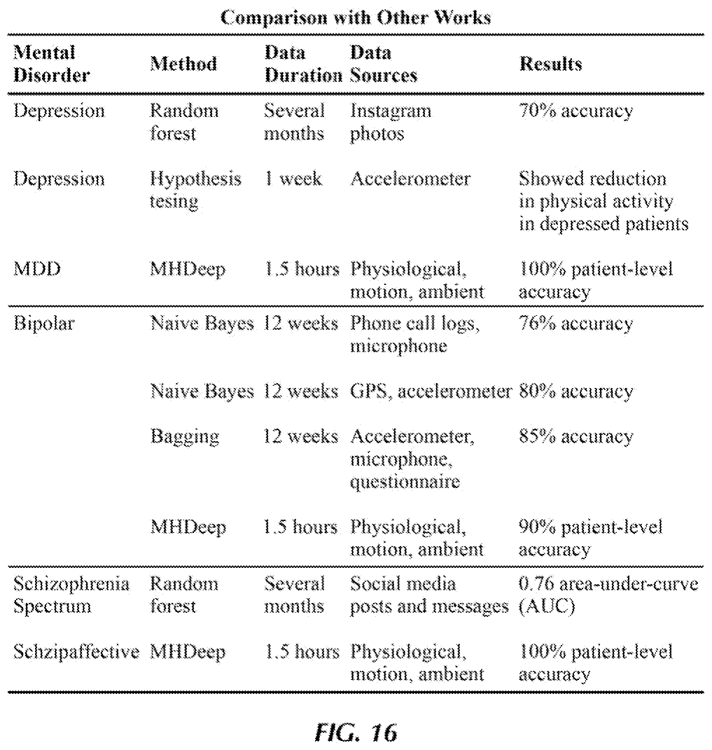

The results of MHDeeps are also compared with other related works on detecting mental health disorders. The results are presented in the table in FIG. 16. Note that since these works use different data sources and solve different problems, the goal of this comparison is only to highlight a few related works on the use of machine learning for identifying mental health disorders. For each study, the method, the duration of data collection, the sources of data used, and the main result are reported. As can be seen, MHDeep is the only approach that uses only 1.5 hours of data from each individual to identify the mental disorders. In addition, contrary to other works mentioned here, MHDeep does not rely on manual feature engineering from various data sources and directly works on the raw sensor data.

### CONCLUSION

MHDeep combines NNs with WMSs to identify various mental health disorders. Although several works address mental health problem detection using machine learning, MHDeep is a solution that focuses on an easy-to-use system that can monitor the daily mental health state of the user through their physiological signals, as well as motion and ambient information. The diagnostic decisions can be sent to a health server from where medical professionals can access the information. This can enable them to quickly intervene during severe episodes of the disorder.

Many mental disorders, such as depression, have different stages with different severities. The progress of such mental health problems can impact the patient's life and health in different ways. As a result, it may be useful if the model can predict disease progression over time. Embodiments of the disclosed framework can be extended to predict the progress of mental health disorders by utilizing longitudinal WMS data collected in the training stage. Furthermore, by accumulating more data from each individual, patient-specific models can be synthesized that are specifically configured based on their data. Such models can be obtained by fine-tuning the trained general models based on accumulated data of the specific patient.

As such, generally disclosed herein are embodiments for a framework called MHDeep that combine data obtained from WMSs with the knowledge distillation power of NNs for continuous and pervasive identification of at least three main mental health disorders: schizoaffective, major depressive, and bipolar. MHDeep uses a synthetic data generation module to address the lack of large datasets. The NN models are trained by using iterative network growth and pruning to learn both the weights and architecture during the training process. MHDeep was evaluated based on data collected from 74 individuals. It achieves patient-level accuracy of 100%, 100%, and 90.0%, using 40, 16, and 22 minutes of data collected in the inference stage, for classification between healthy and schizoaffective disorder individuals, healthy and major depressive disorder individuals, and healthy and bipolar disorder individuals, respectively. The MHDeep models were also shown to be computationally efficient. Thus, MHDeep can be employed for pervasive diagnosis and daily monitoring while offering high computational efficiency and accuracy.

It is understood that the above-described embodiments are only illustrative of the application of the principles of the present invention. The present invention may be embodied in other specific forms without departing from its spirit or essential characteristics. All changes that come within the meaning and range of equivalency of the claims are to be embraced within their scope. Thus, while the present invention has been fully described above with particularity and detail in connection with what is presently deemed to be the most practical and preferred embodiment of the invention, it will be apparent to those of ordinary skill in the art that numerous modifications may be made without departing from the principles and concepts of the invention as set forth in the claims.

1. A machine-learning based system for identification and monitoring of a mental health disorder, comprising one or more processors configured to interact with a plurality of wearable medical sensors (WMSs), the one or more processors configured to:
receive physiological data from the WMSs;
train at least one neural network based on raw physiological data augmented with synthetic data and subjected to a grow-and-prune paradigm to generate at least one mental health disorder inference model; and
output a mental health disorder-based decision by inputting the physiological data from the WMSs into the at least one mental health disorder inference model.

2. The machine-learning based system of claim 1, wherein the physiological data comprises at least one of galvanic skin response, skin temperature, inter-beat interval, and three-way acceleration received from a smartwatch.

3. The machine-learning based system of claim 1, wherein the physiological data comprises at least one of motion patterns, ambient temperature, gravity, acceleration, and angular velocity received from a smartphone.

4. The machine-learning based system of claim 1, wherein the one or more processors are further configured to synchronize and normalize the physiological data from the WMSs.

5. The machine-learning based system of claim 1, wherein the grow-and-prune paradigm comprises the at least one neural network growing at least one of connections and neurons based on gradient information and pruning away at least one of connections and neurons based on magnitude information.

6. The machine-learning based system of claim 5, wherein growing at least one of connections and neurons based on gradient information comprises adding connection or neuron when its gradient magnitude is greater than a predefined percentile of gradient magnitudes based on a growth ratio.

7. The machine-learning based system of claim 5, wherein pruning away at least one of connections and neurons based on magnitude information comprises removing a connection or neuron when its magnitude is less than a predefined percentile of magnitudes based on a pruning ratio.

8. The machine-learning based system of claim 1, wherein the grow-and-prune paradigm is iterative.

9. The machine-learning based system of claim 1, wherein the one or more processors are further configured to generate the synthetic data using a Gaussian mixture model.

10. The machine-learning based system of claim 1, wherein the one or more processors are further configured to label the synthetic data using a machine learning model.

11. The machine-learning based system of claim 1, wherein training the at least one neural network further comprises pre training the at least one neural network with the synthetic data.

12. The machine-learning based system of claim 1, wherein the mental health disorder comprises at least one of schizoaffective disorder, major depressive disorder, and bipolar disorder.

13. A machine-learning based method for mental health disorder identification and monitoring utilizing one or more processors configured to interact with a plurality of wearable medical sensors (WMSs), the machine-learning based method comprising:
receiving physiological data from the WMSs;
training at least one neural network based on raw physiological data augmented with synthetic data and subjected to a grow-and-prune paradigm to generate at least one mental health disorder inference model; and
outputting a mental health disorder-based decision by inputting the physiological data from the WMSs into the at least one mental health disorder inference model.

14-24. (canceled)

25. A non-transitory computer-readable medium having stored thereon a computer program for execution by a processor configured to perform a machine-learning based method for mental health disorder identification and monitoring, the machine-learning based method comprising:
receiving physiological data from a plurality of WMSs;
training at least one neural network based on raw physiological data augmented with synthetic data and subjected to a grow-and-prune paradigm to generate at least one mental health disorder inference model; and
outputting a mental health disorder-based decision by inputting the physiological data from the plurality of WMSs into the at least one mental health disorder inference model.

26-36. (canceled)
